(19) United States
(12) Patent Application Publication
(10) Pub. No.: US 2026/0134962 A1
(43) Pub. Date: May 14, 2026

(54) GENERATION OF AND SEARCH FOR MEDICAL REFERENCE IMAGE DATA

(71) Applicant: Smart Reporting GmbH, Munich (DE)

(72) Inventors: Su Hwan KIM, Munich (DE); Alvaro SANCHEZ MOSCOSA, Munich (DE)

(21) Appl. No.: 19/120,486

(22) PCT Filed: Oct. 17, 2022
(86) PCT No.: PCT/EP2022/078879

§ 371 Date: Apr. 11, 2025

(51) Int. Cl. G16H 15/00; A61B 5/00; G16H 30/20

(52) U.S. Cl. CPC G16H 15/00; A61B 5/7425; G16H 30/20

(57) ABSTRACT

A computer-implemented method, computer-system, computer-program product, computer-readable storage medium, and data carrier signal for associating findings report entries of a structured medical findings report concerning medical image data and comprising at least one findings report entry, and medical image data or one or more subsets of medical image data are proposed. To this extent the medical image data displayed to a user are temporarily synchronized with the entries of a structured medical findings report and associated this way. Furthermore, a computer-implemented method, computer-system, computer-program product, computer-readable storage medium, and data carrier signal for providing medical reference image data to a user during creation of a structured medical findings report are proposed. One or more databases comprising information of the association of finding report entries and medical images data as well as the entries of a structured medical image report being created are used for performing this database query.

### TECHNOLOGICAL FIELD

The present invention relates to a computer-implemented method, a computer system, computer program product, computer-readable storage medium, and data carrier signal according to any of the preambles of claims 1, 6, 10, 11 and 13 to 15, respectively.

### BACKGROUND

In the field of radiology, describing and interpreting visual patterns in imaging examinations (e.g., X-ray, CT, MRI) is one of the most important activities of physicians. Even though Al algorithms are increasingly used to automate image analysis, the assessment of their accuracy and contextualization remains a core task of the radiologist.

The ability of radiologists to recognize and classify image findings is trained by the repeated correlation of image findings with descriptors and diagnoses provided either by more experienced, skilled radiologists or by subsequent, more accurate diagnostic methods (e.g., a tissue biopsy may confirm whether a tumor observed in an image study is benign or malignant).

However, the learning process of repeated correlation described above is an ongoing one. That is, the radiologist trains his eye as well as expands his experience through his daily work. As part of this learning process, in which the clinician acquires medical knowledge and expertise that can be applied to future patient cases, historical patient cases are a valuable resource. However, also in the case of ambiguous or difficult-to-interpret findings, the radiologist usually searches for images of previous cases as well as relevant background information and compares them with the images of the current case.

While collected evidence from clinical trials is more generalizable than individual case reports in many contexts, in some areas observation and internalization of typical patterns from previous patient cases can be very useful to correctly identify similar new cases. Reference image data of similar, earlier patient cases are a valuable aid in this regard. However, it is sometimes challenging for radiologists to find adequate reference image data.

In the past, search engines for radiology images were developed to facilitate learning from previous annotated cases. Existing search engines for radiology use text and terms or image snapshots as input to the search algorithm (e.g., clinical decision support and deep learning-based tools).

However, this search process is decoupled from the core workflow of radiology reporting. In fact, the user has to start a separate workflow when searching reference image data and try to find relevant images based on manually entered search terms.

For example, WO 2019/190518 A1 describes a computer system configured as a search tool for an image search within a reference library in which at least a portion of the images are associated with metadata containing clinical information.

U.S. Pat. No. 11,126,649 B2 describes another computer-based system for identifying and referencing radiological images similar to a query image. In this process, a similarity score is generated between the query image and each candidate image.

US 2008/0027917 A1 describes a computer-based system for searching for an image of interest in a large number of images. The search is based on a database of semantic image representations and a set of queries associated with the semantic image representations. A semantic search engine then compares the plurality of images with the semantic image representations associated with a query selected from the set of queries.

A further challenge in addition to the search for adequate reference image data is the creation of annotated image datasets that can possibly serve as reference image data. This sometimes requires a great deal of manual effort. In this context, annotations of image data are very valuable and can be used to train Al algorithms or to quickly find relevant images during clinical routine (e.g., when the radiologist wants to quickly find an important finding to show to the referring physician). However, the generation of annotated image datasets is usually time-consuming, as image annotation is often done manually. In addition, image annotation is also decoupled from the routine workflows of radiologists. Thus, although radiologists evaluate the images and document the corresponding descriptors in the report, image and descriptor cannot be associated at a granular level. To generate annotated image datasets, dedicated scientific staff usually have to spend many hours manually entering these associations into a database.

In CN 110931095 A, a radiologist browses DICOM images in an image browser. If the radiologist identifies a focus in an image and makes a marker in this image (with associated label shape, coordinates and measured values), a window for editing the focus parameters opens, by means of which an entry belonging to the focus can be made in a structured report. The radiologist can thus edit various parameters associated with the focus in order to create marked structural content. The UID (Unique Identifier Value), the serial number and the image number of the labeled image as well as all labeled shapes, coordinates and measurement values of the labeled image are additionally recorded. Based on the UID, the labeled image and the generated marked structured content can then be added to a corresponding structured report.

Structured reports of findings have been known for some time. Structured (medical) reports comprise user-generated report entries/findings entries and a machine-readable hierarchical data structure. Each user-generated report entry is assigned at least one element of the data structure by a computer. Optionally, the semantic meaning of the entry or data structure element may be uniquely encoded, e.g., by an ontology system and corresponding ontology codes.

Both the image search and the generation of annotated image datasets as well as the generation of corresponding databases of such annotated image datasets are therefore complex, often difficult to implement and, above all, not integrated into the routine workflow of the radiologist.

### SUMMARY OF THE INVENTION

It is therefore desirable to further improve the generation of annotated image data sets and the associated databases, as well as the search for medical reference image data and, optionally, further medical reference data, and, in particular, to better integrate these operations into the physician's workflow.

The invention solves this problem with the computer-implemented methods according to claims 1 and 6, the systems according to claims 10 and 11, in the computer program product according to claim 13, the computer-readable storage medium according to claim 14 and the data carrier signal according to claim 15.

Some aspects of the present invention include associating selected entries of a medical structured findings report and medical image data or subsets of these image data. In this way, medical image data are automatically annotated. According to this aspect of the invention, making an entry in a structured medical report (report entry) triggers determining which medical image data or subset of the medical image data was displayed within a particular time window before, during, and/or after the report entry was generated. This approach may, e.g., assume, that evaluation of an image and the entry of the associated structured findings element is usually carried out in close temporal connection. The generated findings report entry and the medical image data or its subset determined in this way are then associated with each other. Herein, the term “association” covers not only the abstract logical process of association, but also its implementation at the level of concrete storage of the corresponding data and information. Examples of these concrete implementations are described below in the context of some embodiments of the invention.

The storage of data and information includes storing of the implemented associations in at least one database. The possibilities of this storage are described below in the context of some embodiments of the invention.

Finally, other aspects of the invention include searching for reference image data in one of the described databases. In particular, this search can be performed in the context of the creation of a structured medical findings report. For example, it can be embedded in the software environment used for generating findings reports. Examples of the implementation of this search are described below in the context of some embodiments of the invention.

One first aspect of the invention relates to a computer-implemented method for associating findings report entries of a structured medical findings report concerning medical image data and comprising at least one findings report entry, and medical image data or one or more subsets of these medical image data. Optionally, this aspect may also relate to generating a database comprising associations of findings report entries of structured medical reports and one or more subsets of medical image data. For the method one or more subsets of the medical image data are displayed in an image display environment for a user to inspect them. The user creates a medical findings report entry in the structured medical findings report. A creation time attributable to the creation of the medical findings report entry is then automatically recorded/captured, e.g., by the computer system. Further, the one or more subsets of medical image data that are displayed within a predetermined time period attributable to the recorded creation time are automatically determined. Finally, the one or more subsets of the medical image data determined in preceding steps and the created medical findings report entry are automatically associated. Alternatively, or additionally, image identification data enabling the determined one or more subsets of the medical image data to be located and the created medical findings report entry are automatically associated. In an additional, optional step, the made association is automatically stored in a database.

In some of embodiments of this aspect, the predetermined time period attributable to the recorded creation time comprises the recorded creation time.

In some other of embodiments of this aspect, in addition, the determined one or more subsets of medical image data is the most recently displayed one or more subsets of medical image data within the predetermined time period attributable to the recorded creation time.

In some embodiments of this aspect, the displayed medical image data was generated using at least one of the following modalities: Magnetic resonance imaging (MRI), computer tomography (CT), X-ray, ultrasound (US), positron emission tomography (PET), single photon emission computer tomography (SPECT), scintigraphy, and electrograms like Electroencephalography (EEG), Electroneuronography (ENG), Electromyography (EMG) and/or Electrocardiogram some embodiments of this aspect, the subsets of the medical image data are at least one of one or more slices of a set of tomographic image data, one or more regions of interest in one or more medical images or one or more parts of an electrogram.

In some embodiments of this aspect, additionally, setting parameters used for displaying the determined one or more subsets of the medical image data in the image display environment are automatically determined, and, additionally are automatically associated with the created findings report entry. Such setting parameters are used to determine the appearance of displayed graphical information, especially images, i.e., how they are presented in a display.

In some embodiments of this aspect, upon automatic storage of the association, the database stores at least one of the structured medical findings report, the findings report entry, findings report identification data enabling the findings report entry to be located, a findings identifier, and an ontology code uniquely identifying the findings. In addition, the database stores at least one of the determined one or more subsets of the medical image data associated with the findings report entry or image identification data enabling the determined one or more subsets of the medical image data to be located.

In some embodiments of this aspect, automatically storing the association in the database further includes storing at least one of patient-identifying data and medical findings report-identifying data. In some embodiments of this aspect, upon automatic storage of the association in the database, the setting parameters used for displaying the determined one or more subsets of the medical image data in the image display environment associated with the medical findings report entry are also stored in the database.

According to a second aspect of the invention, a computer-implemented method for providing medical reference image data during the generation of a structured medical findings report on a patient including one or more medical findings report entries is proposed. For this method a reporting environment for creating structured medical findings reports is provided to a user. Further an automatic or semi-automatic search for medical reference image data is provided to the user during the creation of a medical findings report entry in the structured medical findings report. The search is performed in a database generated according to the first aspect of the invention, uses as input parameters information retrievable from the structured medical findings report currently being generated and, in particular, the medical findings report entry. Additionally, or alternatively, further information retrievable from other sources than the structured medical findings report currently being created can be used as input parameters. Based on the input parameters, the search identifies in the database subsets of the medical image data to provide them as output medical image data. Finally, the output medical image data determined by the automatic or semi-automatic search are provided as reference image data.

In some embodiments of this aspect, the provided automatic or semi-automatic search additionally searches for reference medical findings reports associated with the reference medical image data.

In some further embodiments of this aspect, the steps of the second aspect are combined with any of the steps of the first aspect.

A third aspect of the invention relates to a computer system for associating medical findings report entries of a structured medical findings report concerning medical image data and comprising at least one medical findings report entry, and one or more subsets of the medical image data. The system comprises at least one processor and at least one memory having stored thereon instructions. Optionally, the system comprises at least one database. When the instructions are executed on the at least one processor, they cause the computer system to provide means for creating structured medical findings reports, means for displaying medical image data, means for recording a creation time attributable to the creation of a medical findings report entry in the structured medical findings report, means for determining one or more subsets of the medical image data displayed within a predetermined time period attributable to the recorded creation time, means for associating the determined one or more subsets of the medical image data or image identification data enabling the determined one or more subsets of the medical image data to be located, and the created medical findings report entry. Optionally, when the instructions are executed on the at least one processor, they cause the computer system to provide means for automatically storing the associations in a database.

A fourth aspect of the invention relates to a computer system for providing medical reference image data during the generation of a structured medical findings report on a patient including one or more medical findings report entries. The system comprises at least one processor, a least one database generated according to the first aspect of the invention and at least one memory having stored thereon instructions. The instructions, when executed on the at least one processor, cause the computer system to provide means for creating structured medical findings reports, means for displaying medical image data, means for providing an automatic or semi-automatic search for medical reference image data when creating a medical findings report entry in the structured medical findings report, wherein the medical findings report entry is related to findings in medical image data of the patient. The search is performed in the at least one database, and uses as input parameters information retrievable from the structured medical findings report currently being created, in particular from the medical findings report entry. Additionally, or alternatively, further information retrievable from other sources than the structured medical findings report currently being created can be used as input parameters. The search identifies, based on the input parameters, in the database subsets of the medical image data to provide them as output medical image data. Further, the instructions, when executed on the at least one processor, cause the computer system to provide means for providing the output medical image data determined by the automatic or semi-automatic search as reference image data. Optionally, the system may comprise means for displaying medical image data and displays the reference image data in the means for displaying medical image data.

A fifth aspect of the invention relates to a computer program product comprising instructions for execution on at least one processor that, when executed, cause the processor to perform the steps of any one of the first or second aspect of the invention.

A sixth aspect of the invention relates to computer-readable storage medium on which the computer program product of the fifth aspect of the invention is stored.

A seventh aspect of the invention relates to a data carrier signal comprising the computer program product of the fifth aspect of the invention.

Aspects and embodiments of the invention are discussed below. The details and resulting advantages of the invention will become apparent from the following description. Where appropriate, reference is made to the corresponding drawings, which show preferred embodiments of the invention for illustrative purposes. However, these embodiments do not necessarily represent the full scope of the invention.

### BRIEF DESCRIPTION OF THE DRAWINGS

FIG. 1 illustrates a system according to the invention comprising a reporting environment, an image display environment and a report database containing medical structured reports;

FIG. 2 illustrates a system according to the invention comprising reporting environment, an image display environment and a database comprising meta information;

FIG. 3 illustrates a system according to the invention comprising reporting environment, an image display environment and an image database comprising medical image data or subsets thereof;

FIG. 4 provides a flow chart of actions for performed in some examples of embodiments of the invention in order to associate findings report entries of a structured medical findings report and subsets of medical image data;

FIG. 5 provides a flow chart of actions for performed in some examples of embodiments of the invention in order to provide reference image data during the creation of a structured medical findings report

FIG. 6 provides a schematic overview of the interactions between the reporting environment, an image display environment and a database in order to search the database for reference image data.

FIG. 7a-e illustrates the timing of a reporting event, e.g., the generation of an entry in a medical structured report, and the relevant temporal interval of viewing/showing medical images in the image display environment for some examples of embodiments of the invention;

### DETAILED DESCRIPTION

The order of the steps described below can be varied if necessary. In addition, the individual steps can be performed individually or as combinations of a selection of the individual steps.

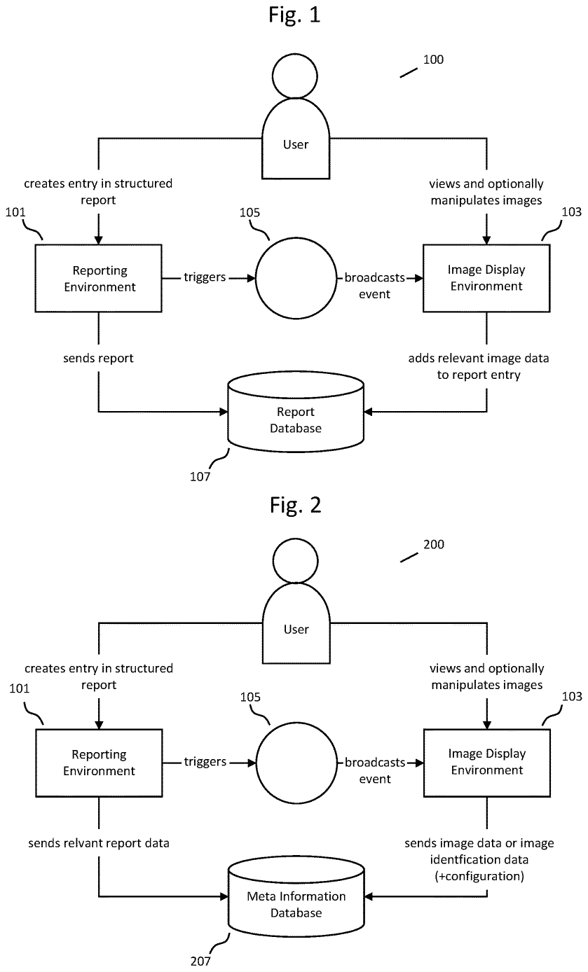

FIGS. 1-3 illustrate examples of embodiments of the invention for associating findings report entries and medical image data/subsets of the medical image data. The embodiments shown therein each include a system comprising at least one of the following: a reporting environment 101, an image display environment 103, a hub 105, and a database 107, 207, 307. In the context of this application, the term environment includes all programs executable by a computer (computer programs), in particular software, software environments, and software components.

Examples of a reporting environment include computer programs suitable for generating medical structured reports and, in particular, those specifically configured to do so.

Examples of image display environments include computer programs suitable for displaying digital image data, and in particular, those specifically configured to do so. In some embodiments, these computer programs may be specifically configured for displaying and viewing medical images. For example, the computer program may be a DICOM viewer.

Examples of a hub include computer programs, including in particular one or more systems of software units or components, configured to detect events in a first computer program and to communicate the occurrence of a detected event to one or more second computer programs, if applicable. In some embodiments, a hub may also additionally serve to exchange information and data between different computer programs. In particular, communication may be controlled by existing interoperability standards. One example of such a standard is the FHIRcast standard, that enables a standardized way of performing context synchronization. Application Programming Interfaces (APIs) based on such a standard allow synchronization of disparate healthcare applications'user interfaces in real time.

The databases described below are arranged for storing and searching computer-readable information. In some embodiments, this information includes structured medical findings reports, digital medical image data or at least subsets of medical image data, and/or meta-information about findings reports, individual entries of findings reports, medical image data, or combinations thereof.

The term medical image data includes all visualized medical information. In some examples this includes Electrograms like EEG, ENG, EMG and/or ECG. In other examples this additionally or alternatively includes sets of medical images, single medical images as well as snapshots of medical images. In the context of this application, snapshots are to be understood as reproductions of a part of the visualized medical information. For example, individual medical images from a set of images or reproductions of the display of the medical image data visible to a user, e.g., a screenshot of the image data as displayed at a particular time. For example, a snapshot may comprise only a subset of the visualized medical information. For example, in a medical image data set consisting of multiple images (for example, in a tomographic slice imaging), a snapshot may consist of a single one of those images (for example, a single slice). In further examples, a snapshot may comprise only a subset of all intensity values measured in an image. Medical image data may consist of one or more subsets of medical image data.

The term subset(s) of medical image data includes at least one of one or more slices of a set of tomographic image data, one or more regions of interest in one or more medical images or one or more parts of an electrogram. E.g., if the medical image data are a set of tomographic images, e.g., a 3D-MRI-image, a subset may be formed by one or more medical images/slices or even one or more pixels or voxels within one image/slice, e.g., one or more regions of interest. If, in another example the medical image data is an X-Ray- or US-image a subset may be the respective image itself or a group of pixels of the image. If the medical image data is an electrogram, e.g., an EEG, a subset may be a part of the electrogram, e.g., a portion of the electrogram measured in units of length such as millimeters, centimeters, meters, or units of time such as milliseconds, seconds, minutes, or frames, or any other arbitrary units. In some embodiments, the report entries are associated to the entire medical image data, e.g., an entire image series the radiologist is reporting on. Thus, in these embodiments the association is based on the information to which medical image data the structured medical findings report corresponds and a predetermined time period attributable to the creation time of an entry is not mandatory.

In some embodiments, the meta-information comprises at least one of partial information and/or complete information regarding structured medical findings reports, digital medical image data, one or more patients, one or more groups of patients, one or more medical facilities, one or more medical devices, and one or more findings or medical conditions. The meta-information may be in coded or non-coded form. For example, the meta-information may include information regarding the content of the findings reports or entries thereof and/or the storage location thereof. Additionally, or alternatively, the meta-information may include information about the content of the medical image data, setting parameters used when displaying and viewing the image data, and/or image acquisition details (e.g., modality, location, date, protocol, etc.). Additionally, or alternatively, the meta-information may include information about the storage location of the medical image data, identification of portions of the medical image data within an image data set, and ranges of pixels/voxels within individual images.

The computer programs described above may each be executed on separate hardware (e.g., on hardware that is physically or virtually separated from each other), or part of the software described above may be executed on a first hardware and another part may be executed on a second, third, . . . , or Nth hardware. Moreover, the computer programs described above may also be executed in a distributed system. For example, the reporting environment, the hub, and/or the image display environment may each comprise multiple separate software subunits and execute them in a distributed manner on multiple processors of one or more computers or in a cloud.

The systems of some of the embodiments described receive input from a user, for example, a physician, particularly a radiologist. Hardware components such as a keyboard, mouse, trackpad, writing/drawing pad, touchscreen, camera, or microphone may be used to capture the user-side input, as well as corresponding software to convert the input via this hardware into usable input. The input includes, for example, user-side input for generating an entry in the structured medical findings report or for annotation and/or measurement in the viewed image, and for configuring the setting parameters of the image display environment.

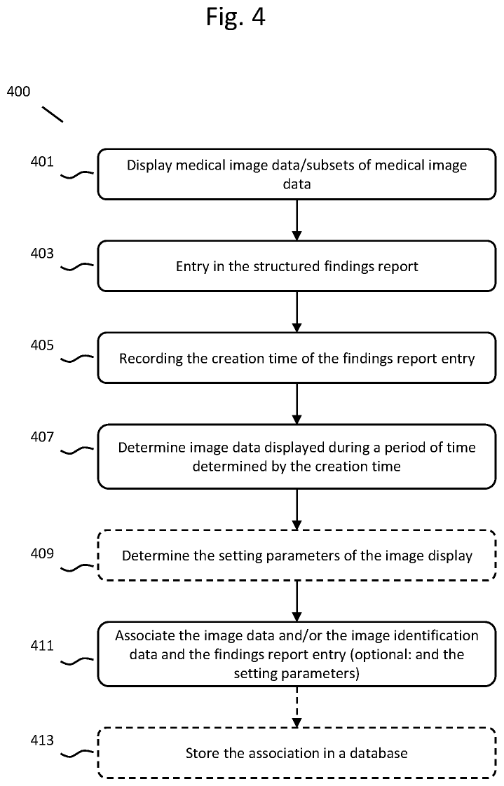

FIG. 4 shows a flowchart 400 illustrating the association of findings report entries and medical image data according to some embodiments. Not all of the steps shown need to be performed in the sequence described. Rather, the order of all or individual steps can be changed, if appropriate.

In a step 401, medical image data or at least one subset of medical image data is displayed to a user.

In particular, this may be medical image data of at least one of the following image modalities: Magnetic resonance imaging, computed tomography, X-ray, ultrasound, positron emission tomography, scintigraphy, and electrograms like Electroencephalography, Electroneuronography, Electromyography and/or Electrocardiography.

In particular, it will be common for a user to view images of a tomographic imaging (e.g., MRI, CT, PET, . . . ). In tomographic imaging, numerous 2D tomographic images, also called image slices, are created for a 3D object, where each of the 2D tomographic images represents a cross-sectional plane of the 3D object. A medical image dataset consisting of a tomographic image of a 3D object then contains a large number of 2D slice/tomographic images, each of which is uniquely identifiable as a separate image. When reviewing such image data, the user scrolls through this series of images/slices to view the different (slice) planes of the 3D object.

The user then examines the individual images, for example, by reviewing one image at a time.

In connection with the displayed image data, the user makes a findings report entry in step 403. For example, he or she enters an observation made in the image data as a findings report entry.

Triggered by step 403, the time at which the findings report entry was made ((findings report entry) creation time) is determined in step 405. In some embodiments, the findings report creation time may be determined based on, for example, the time at which at least one of the above input means is used. For example, a start time, an end time, a midpoint, or any other time during the use of said input means may be used to determine the creation time.

In step 407, it is then determined which image data was displayed to the user within a specific time interval. This time interval represents a predetermined period of time that can be assigned to the recorded creation time. The time interval can be defined differently as illustrated in FIG. 7 a-e on the basis of a time axis. In FIG. 7a, for example, it starts and ends before the report creation time. In the examples of FIGS. 7b-7d, it includes the report creation time, i.e., the creation time is its end point (FIG. 7b), its start point (FIG. 7d) or lies between its start and end point (FIG. 7c). The predetermined period of time attributable to the recorded time of creation thus comprises the recorded creation time in this case. In particular, the determined creation time can be a boundary of the interval (beginning or end), as in FIGS. 7b and 7d. However, as in the example of FIG. 7e, the period of time can also lie after the time at which the findings report is created. In the case of multiple sets of image data being displayed at the same time, e.g., in several windows, not only the images being displayed within the given time interval are determined, but also those being focused during the time interval. E.g., only those image data are determined as relevant that are displayed in an active window or active section of a window during the defined time interval. Once the relevant image data or information to locate the relevant medical image data, i.e., image identification data, are determined, they are made available, e.g., by broadcasting them.

In some embodiments, only the most recently displayed subsets of the medical image data within the time interval, i.e., the predetermined period of time attributable to the detected creation time, is determined. This may be useful, for example, in the case where several different image data are subsequently displayed within the time interval.

In some embodiments, the time interval has a length of 60, 30, 20, 10, 5, 4, 3, 2, 1 or 0.5 seconds. In some embodiments, the creation time point may be at any time point within the interval as previously described, and in particular may form the start or end time point of the interval, that is, the start or end time point may have a temporal distance of 0 seconds from the creation time point. In other embodiments, both the start time and the end time of the interval have a temporal distance from the creation time. For example, the end time point of the interval may be 60, 30, 20, 10, 5, 4, 3, 2, 1 or 0.5 seconds before the creation time point. In other examples, the start time of the time interval may be 60, 30, 20, 10, 5, 4, 3, 2, 1 or 0.5 seconds after the creation time point.

In some embodiments, the time interval, its length, its start point, and/or its end point is determined dynamically. For example, the time interval is determined such that the temporal distance to a previous or a subsequent data input in the reporting environment is not exceeded. For this purpose, for example, the start point for a time interval starting before the creation time can be shifted correspondingly closer to the creation time. Or, the end point for time interval ending after the creation and be shifted correspondingly closer to the creation time. This is intended to prevent incorrect associations of findings report entries and medical image data, where, for example, the image data belonging to a previous findings report entry is incorrectly associated with subsequent findings report entry. For example, it can be implemented that the upper limit of the time interval (with respect to the creation time) may extend at most to the time of a previous data entry in the reporting environment, or the limit of the time interval (with respect to the creation time) may extend at most to the time of a subsequent data entry in the reporting environment.

In some embodiments, in step 407, image identification data is also automatically determined for the image data displayed in one of the above time intervals. Image identification data may be any data suitable for identifying and locating one or more image data from a plurality of image data. Additionally, or alternatively, image identification data may include one or more points within an image. For this purpose, any information can be used that uniquely identifies image data and/or an image area, i.e., an area within an image, in the sense of image coordinates and thus makes it locatable. Points and areas in an image may, e.g., be represented by individual pixels and/or voxels, or groups of pixels and/or voxels.

In some embodiments, the image viewer prompts the user to select one point within a current 2D image to locate the finding in more detail-this workflow is only semi-automatic but provides more useful annotations. Annotations may be a pointer, ROI mark, polygon, etc.

The medical image data or information to locate the relevant medical image data, i.e., image identification data, are made available, e.g., by broadcasting them.

In some embodiments, where individual images or slices are identified, a serial and/or an image number is used for a specific image, e.g., a specific slice and/or a specific time point in a 3D or 4D image dataset. For example, image identification data may be a UID (unique identifier) of a DICOM image. The UID is a unique identifier value for each DICOM image and is automatically formed after a DICOM image of a patient is acquired. The UID value contains series and image numbers, which are used to uniquely identify the specific image series and each individual image. In addition, further information, such as the hospital, can be determined from the DICOM UID. In other embodiments, snapshots of the determined images are generated in addition or as an alternative to the image identification data described above. In further embodiments, the memory location/address of the image data serves as the image identification data. In further embodiments, combinations of the storage location and image and/or serial numbers are used as image identification data.

In further embodiments, 3D image identification data is determined which also contains coordinates within a two-dimensional image. These then correspond to one or more pixels/voxels. This additional information in the form of image coordinates can be determined, for example, by interactions of a user with the image in the image area identified by the coordinates. For example, if a user marks individual pixels/voxels within a 2D image, the coordinates of the pixels/voxels in question become part of the image identification data. The user may make such markings manually, semi-automatically, or fully automatically, for example, as part of a segmentation process. In further examples, the user makes measurements within specific pixels/voxels, for example, of image intensity or dimensions of a region of interest (ROI), specific thicknesses or angles, or physiological parameters such as flow, density, relaxation time, diffusion coefficient, or perfusion. In another example, the user annotates the pixels/voxels in question. All these interactions can be used to identify specific pixels\voxels and, therefore, to determine and provide image identification data.

The image identification data is interesting for user interactions, for example, because it allows the user to access original image series directly. In this case, a relevant image of an image series would be opened first (e.g., the image that is focused exactly at the time of the “findings report entry”). However, the user could still access all or at least a part of the further images (i.e., additional images) in the image series if necessary. Also, based on the original image data, the user could adjust the setting parameters to display the images. Especially if the user has access to the original image series, e.g., if the images are from the same institute/hospital, this possibility is advantageous. This can be a performance advantage, because it may be more efficient and faster for a computer to query the local database than to download the same images from another database again.

In addition to, or as an alternative for, using the creation time in a corresponding time interval to associate medical findings and medical image data, the creation of an annotation in medical image data, e.g., by performing a measurement (e.g., diameter, gray values in Hounsfield Units-in CT images, etc.), is transmitted, as an event, to the medical findings report and in particular to a specific findings report entry of the report. Besides the content of the annotation, e.g., the results of the measurement, also the annotated image itself, or image identification data for this image, may be transferred to the medical findings report entry. This way, medical findings report entries and medical image data can be associated with each other. This may be further illustrated by the following example. A user measures the diameter of the tumor in the image viewer. The measured value is automatically transferred to the reporting system and in particular the corresponding findings report entry. Then, only the one image in which the measurement was made has to be associated with the measured value received from the reporting system, or the image itself or its image identification data have to be transferred to the reporting system.

In step 411, the findings report entry(s) generated and the image data determined are associated with each other.

In some embodiments, at least one of the determined image identification data or the image data determinable by the image identification data, for example images or snapshots, is added to the findings report entry for this purpose. For example, the data structure of the structured medical findings report may provide corresponding data structure elements configured to store said data.

In further embodiments, portions of the findings report and/or findings report entry, as well as at least one of the image identification data or the determined images, are stored in a separate database for association. Further details of storage in a database will be described later on.

In further embodiments, portions of the findings report and/or findings report entry are added to the particular images for association and stored with them in the corresponding image database. In some of these embodiments, newly established or existing mechanisms for annotating medical image data may be used for this purpose. For example, the findings report entry itself and/or relevant portions thereof may be added to the image(s) as an annotation—for example, the finding in words or coded, e.g., by a corresponding code of a medical ontology.

In some embodiments, the steps for 405-411 are performed only for certain types of findings report entries. For example, they are performed only when the findings report entry does not correspond to a normal finding. In other words, only findings report entries that correspond to a disease condition, pathology, or other deviation from the norm trigger the aforementioned steps.

In some further embodiments, in a step 409, the configuration of the image display environment is additionally determined automatically, i.e., the setting parameters used for displaying or viewing the one or more subsets of the medical image data in the image display environment. These setting parameters are then also associated with the findings report entries in step 411 as described above, and, optionally, be automatically used to set up an image display environment when displaying the stored medical image data. For example, the software used for image viewing or image display may allow image display parameters such as brightness, contrast, white point, or gamma adjustment to be changed. In radiology, for example, it is common to adjust the center and width of the image intensity window, which is also referred to as “windowing.” This involves determining, for example, which section of the measured values (e.g., density values of the Hounsfield scale, or intensity values in MRI) are assigned which gray values from white to black. These values can be stored, for example, as setting parameters.

In some embodiments, in step 413, the association described above is stored in a database. This can be done manually, semi-automatically or fully automatically. This optional additional step thus allows, for example, a database to be created for the search for annotated image data or reference image data.

FIGS. 1-3 schematically show various embodiments 100, 200, 300 that differ in how the association of findings report entries and medical image data and/or image identification data and the storage of this association is implemented. The embodiments shown in FIGS. 1-3 have in common that a user views medical image data or at least one subset of these medical image data in the image display environment 103. Using the reporting environment 101, the user creates an entry in the findings report (findings report entry), e.g., based on an observation and/or measurement made in the image data. The creation of a findings report entry represents an event that triggers the hub 105. In some embodiments, not all findings report entries trigger the hub 105, but only those entries associated with a disease or deviation from the norm, i.e., not with normal findings. The hub 105 or the reporting environment 101 then determines the creation time of the findings report entry and transmits the event and, if applicable, the creation time to the image display environment 103, whereupon the hub 105 or the image display environment 103 determines what medical image data have been displayed or viewed within a given time period in the image display environment 103. As described above in connection with FIG. 7 a)-e), the time period may precede and/or include the creation time point.

In some embodiments the reporting environment 101, the image display environment 103, and/or the hub 105 each include multiple means executed by one or more processors on a computer or distributed on multiple computers.

For example, some of these embodiments include means for creating structured medical findings reports, for example, the aforementioned reporting environment 101, and means for displaying medical image data, for example, the aforementioned image display environment 103. In addition, these embodiments comprise means for detecting a creation time attributable to the creation of a findings report entry in the structured medical findings report, such as a program programmed for this purpose. Such means may, for example, be a modular part of the reporting environment 101 and/or the hub 105. Further, such embodiments include means for determining the displayed medical image data and/or the image identification data already described that identifies the displayed medical image data. Such means may also be implemented in the form of a program programmed for this purpose. Such means may be a modular part of the image display environment 103 and/or the hub 105. Finally, these embodiments include means for associating the determined medical image data and/or image identification data and the created medical report entry/-ies with each other. Again, this may be a program programmed for this purpose. Such means may be a modular part of at least one of the hub 105, the reporting environment 101, and the image display environment 103.

Some of these embodiments further comprise means for determining the setting parameters of the image display environment 103 described above, which may then, in the manner described above, also be associated with the findings report entries and/or medical image data and, if necessary, stored in a database. This may again be a program programmed for this purpose. Such means may be a modular part of at least one of the hub 105 and the image display environment 103.

FIG. 1 schematically shows an embodiment in which the associations already described are stored as part of a findings report database 107, i.e., in the database of completed structured medical findings reports. To this end, in some embodiments, relevant image information is stored as part of the data structure associated with the structured medical findings report. For example, the elements of the data structure, such as nodes and leaves of a tree, may include corresponding elements or containers in which image identification data, medical image data associable therewith, and/or setting parameters of the image display environment 103 used to display these images may be stored. In other embodiments, an additional data structure is provided in which the image identification data, the medical image data associable thereto, and/or the setting parameters of the image display environment 103 used to display those images may be stored. This additional data structure may then be stored in a database 107 along with the structured medical findings report.

FIG. 2 schematically illustrates an embodiment in which the associations already described are stored as part of a database 207 for the aforementioned meta-information. In some of these embodiments, the meta-information includes, in coded or non-coded form, information regarding the content of the findings reports or entries therein and/or the location thereof. Additionally, or alternatively, the meta-information may include, in coded or non-coded form, information regarding the content of the medical image data, setting parameters used in displaying and viewing the image data, and/or the storage location thereof, identification within an image data set, and areas within individual images. For example, the database 207 may include pointers to storage locations of findings report entries or portions thereof and/or medical image data or corresponding image identification data. Further, the database may comprise a mixture of meta-information and the data itself, e.g., meta-information about the medical image data and the findings report entries themselves, or meta-information about the findings report entries and the image data itself. In one embodiment, a database entry includes, a name or designation of the finding and image identification data, e.g., an image ID number and/or a pointer to the location of the medical images. Further embodiments comprise, alternatively, or in addition, to the finding designation, an ontology code identifying the finding and a designation of the ontology system used thereby. Further embodiments additionally comprise a patient ID and/or a report ID. Further embodiments additionally comprise setting parameters associated with the findings report entry for displaying the medical image data or one or more subsets of them. Alternatively, to the image identification data, the image data itself may also be stored. Thus, the database stores at least one of the structured medical findings report, the findings report entry, findings report identification data enabling the findings report entry to be located, a findings identifier, and an ontology code identifying the findings. The database further stores at least one of the determined medical image data or subsets of it associated with the findings report entry, one or more snapshots of the determined medical image data or subsets of it or image identification data enabling the determined medical image data or subsets of it to be located.

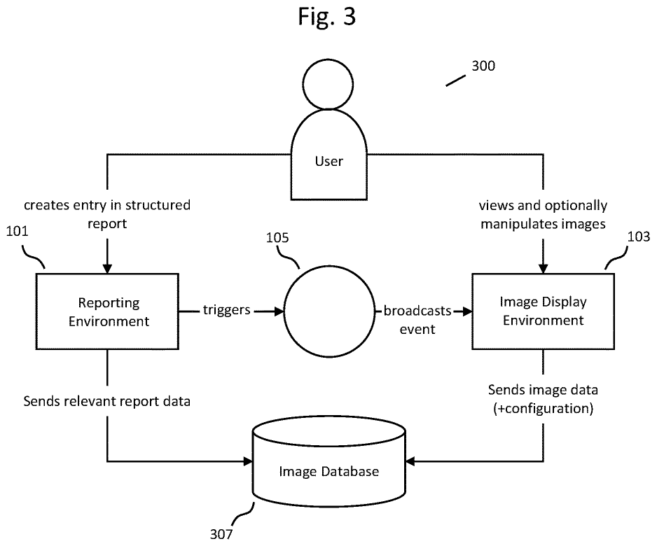

FIG. 3 schematically shows an embodiment in which the associations already described are stored as part of an image database 307. In this case, the medical image data itself or at least parts of the image information contained therein are stored in a database. In addition, at least one of the findings report entry, a findings report designation or a corresponding ontology code, the location of the findings report designation or the findings report, and further information about the patient and/or the findings report are stored. For example, this information can be stored in a manner known to the skilled person in the form of image (data) annotations.

While the methods and systems described so far can be used, e.g., to automatically create an annotated radiology image data set in clinical routine workflows, another important aspect of the invention relates to searching and embedding context-specific image data and clinical background information into a reporting system based on the structured input data. Importantly, the embedded content can be searched and filtered dynamically to suit the context of the individual patient case. Ultimately, this method provides a valuable source of medical education and clinical guidance within the routine reporting workflow.

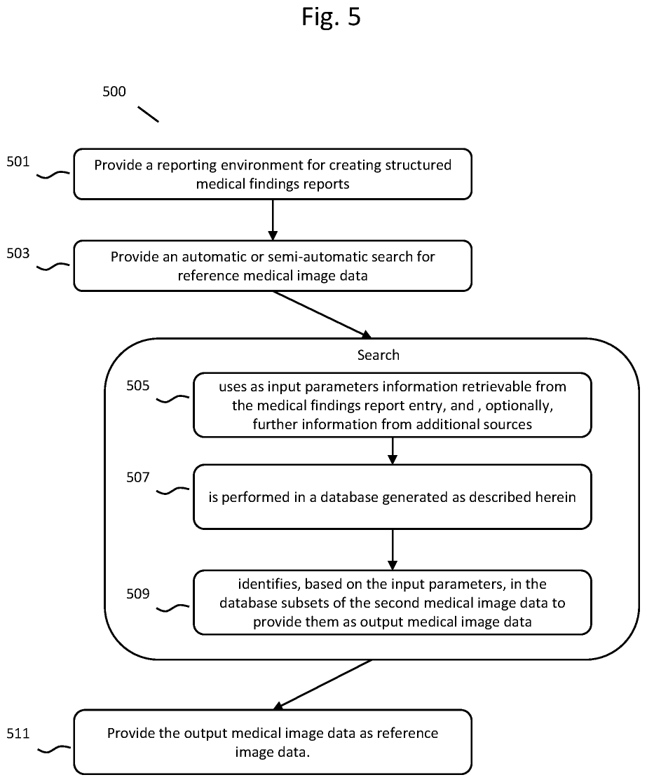

Thus, according to another aspect of the invention and as illustrated in FIG. 5, based on one or more of the described databases 107, 207, 307, a search functionality for medical reference image data, and eventually further medical reference data like reference patient cases and/or reference medical finding reports, is provided. In one embodiment, as step 503 this functionality of an automatic or semi-automatic search for medical reference image data and, optionally, further medical reference data is provided within the reporting environment 105 provided in step 501 for creating structured medical findings reports on a patient including one or more medical findings report entries. A user can thus initiate a search query within the reporting environment and also configure and customize it via the reporting environment. In this way, the user can then initiate as steps 505-509 the search for reference image data, and, eventually, further medical reference data, via the reporting environment 105 so that the search is integrated into the workflow of the reporting environment 105 as fluidly as possible. In further embodiments, the search function is provided (step 503) within an environment separate from the reporting environment 105.

As will be described below, this improves the commonly used procedures, in which, although a great deal of context-specific information on the patient case at hand (e.g., modality, age and gender of the patient, relevant previous diagnoses or suspected diagnoses, other validated image findings) is already available in the system (e.g., PACS/reporting system), this information cannot be readily incorporated into the image search. Instead, the user must start from scratch and try to find relevant images based solely on manually entered search terms.

As a solution, the invention described herein, proposes the following. Regardless of whether the search function is part of the reporting environment 105 or not, it can, in step 505, automatically use the findings report entry currently being processed to configure the search, e.g., as input parameters. Thus, the user does not have to manually configure the search, e.g., by entering search parameters or terms into a search mask. Instead, the information required for the search, e.g., necessary terms and parameters, are automatically extracted by the system from the findings report entry currently being processed or from findings report entries already created, as far as these can be retrieved. In addition, further information retrievable from the findings report and/or further available systems like a picture archiving and communication system (PACS), a hospital information system (HIS), a radiological information system (RIS), a Laboratory Information Systems (LIS), a Document Management Systems (DMS), and/or from Electronic Medical Records (EMR) or header information (e.g., from a DICOM header) may be automatically extracted and used for the search in step 505. The user then does not have to add any information, or at least only some of it. In some embodiments, information retrievable from or the content of the findings report entry is used as input parameters for the search query-in particular when the medical findings report entry is related to findings in medical image data. For example, the naming of the finding or a corresponding ontology code is used to query the corresponding database for matching reference image data. The medical image data determined by the search is then in step 511 displayed in an image display environment, e.g., the image display environment 103 or an additional one. In further embodiments, the search function may automatically adopt additional parameters as search filters. Examples for such parameters are patient's gender, patient's age group, patient's prior diagnoses, patient's prior image findings modality of current examination, etc. The search is then in step 507 performed in a database 107, 207, 307 that was generated in one of the ways described above.

Based on the input parameters, in step 509, the search identifies in the database medical image data or subsets of it to provide them as output medical image data that can be provided as reference image data to a user.

As already pointed out earlier, in some of these embodiments, in addition to the reference medical image data, the search may in step 509 also identify reference patient cases, e.g., in the form of reference medical findings reports and provide them in step 511 to a user. For example, based on identical or similar image-findings combination, similar patient cases can be identified through this. In the end, the search can also be used to access the entire findings reports associated with the images, for example, in order to obtain more case context. If the search result is a patient case from the same institute/hospital, in some of these embodiments, the radiologist could also access additional documents related to the case (e.g., from the hospital information system).

For example, in some embodiments data elements of a reporting template, e.g., visible in a sidebar of the reporting environment 101, turn into selectable filters for image search. For example, selecting “lung tumor” triggers then a search of all lung tumor cases in an annotated image database. Additional selections can be made to add additional query filters. The available filters are equivalent to available report data elements associated with annotated images. In some examples, the images can be further filtered by patient demographics, additional findings, etc. Also, only predefined content may be displayed, e.g., regarding the search options, report entries or images.

In one embodiment, the user activates the search function before starting to create one or more findings report entries, and reference image data is displayed in parallel as the findings report entries are created. The displayed reference image data may be continuously updated on the basis of the findings report entries made up to the respective point in time.

In other embodiments, the user starts the search only after creating one or more findings report entries or portions thereof, and only then is reference image data searched for and displayed. In some of these embodiments, a search function is provided for each of the findings report entries respectively.

As already mentioned, the search is performed by means of one of the databases 107, 207, 307 generated as described above.

As will be discussed later, the methods described above for associating finds report entries and medical image data or subsets of it and storing the associations in one or more databases, and of searching these databases for reference medical images during the creation of a medical findings report, and in particular its entries, can be combined.

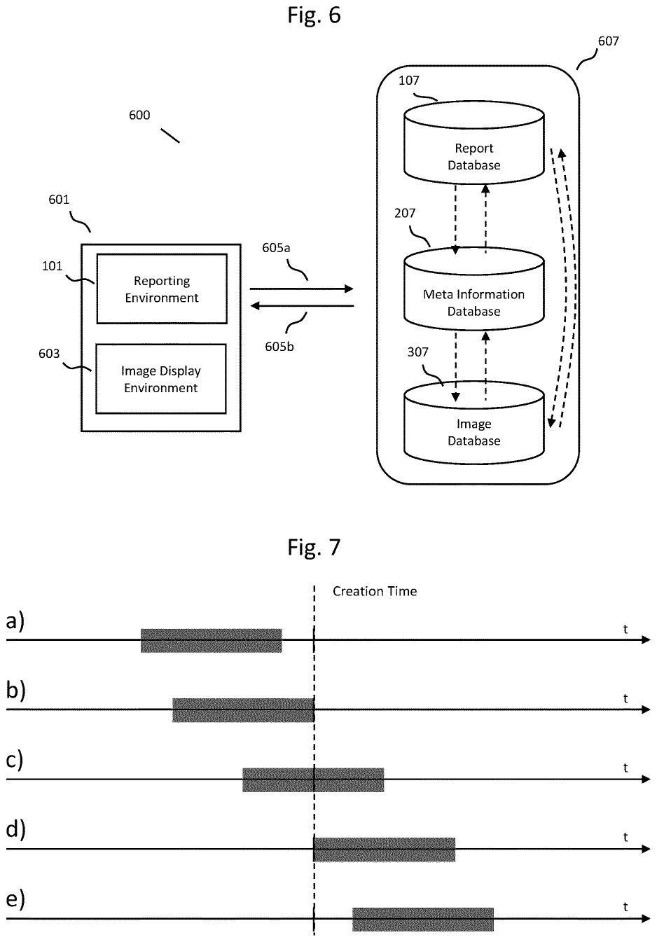

FIG. 6 schematically illustrates interaction between the elements of a system 600 comprising a reporting environment 101, at least one image display environment 603, and one or more databases 607. The at least one image display environment 603 may be the image display environment 103 or may comprise one or more other image display environments. The image display environment 603 may thereby be integrated into the reporting environment 101. The one or more databases 607 thereby comprises at least one of a findings report database 107, a meta-information database 207, and an image database 307. The specific procedure of the specific search query may occur depending on the type of database or types of databases used.

If the database 607 used for the query 605a is set up as in the context of FIG. 1, it is possible, for example, to search for similar or equivalent findings report entries or ontology codes in the findings report database 107 on the basis of the findings designation used for the search query 605a and/or the corresponding ontology code. If image information in the form of medical image data and/or image identification data is available for the hits found, the associated image data can be provided to the user as reference data in a search response 605b in a further step. This can be done within the image display environment 603. If setting parameters are available for the image information, it may be provided that the reference image data is displayed with the image display environment configured with these setting parameters. For example, in some of these embodiments, if the medical image data is stored in the findings report database 107 along with the respective findings report entry, the medical image data is transmitted from the findings report database 107 to the system 601 in a search response 605b. For example, in other embodiments, if image identification data is stored along with the respective findings report entry, the medical image data is located using the image identification data in the image database 307 and transmitted to the system 601 in a search response 605b. In some embodiments, in addition to the image data also the structured medical reports corresponding to the respective reference image data are provided to the user.

If the database 607 used for query 605a is set up as described in the context of FIG. 2, the search query 605a is performed based on meta-information. Similar to what is described in the case of a database according to FIG. 1, this can involve searching for equivalent or identical findings report entries or ontology codes in the meta-information database 207. If image information in the form of medical image data and/or image identification data is available for the hits found, the associated image data can be provided to the user as reference data in a search response 605b in a further step. For example, the image data found at the found storage location, e.g., in the image database 307, is displayed as reference data. This may be done within the image display environment 603. Where setting parameters are available for the image information, it may be provided that the reference image data is displayed configured with these setting parameters. For example, in some embodiments, if finding designations and/or corresponding ontology codes as well as image identification data are stored in the meta-information database 207, the meta-information database 207 is searched for hits based on the finding designations and/or ontology codes, and the corresponding images are located in the image database 307 using the image identification data and transmitted to the system 601 in a search response 605b. In other examples, if the medical image data is stored directly in the meta-information database 207, the medical image data is also transmitted directly from the meta-information database 207 to the system 601 in a search response 605b. In some of these embodiments, the structured medical report corresponding to the transmitted medical image data is, in addition, transmitted to the user. E.g., either the structured medical report is stored in the meta-information database and transmitted directly to the user, or information allowing to locate and identify the corresponding structured medical report is stored in the meta-information database, and, using this information, the structured medical report is retrieved and transmitted to the user.

If the database 307 used for query 605a is set up as in the context of FIG. 3, then based on the search query 605a, the information stored with the image data is searched for associated findings report entries. If the same or equivalent findings or corresponding ontology codes are found therein, the associated image data is provided to the user as reference image data in a search response 605b. This can be done within the image display environment 603. If setting parameters are available for the image information, it may be provided that the reference image data is displayed configured with these setting parameters. In some embodiments, also the structured medical report associated with the provided image data is provided to the user. E.g., information that allows to identify the location of the structured medical report is also stored with the medical image data and used to retrieve the structured medical report in order to provide it to the user.

One aspect of the invention relates to a computer system for associating findings report entries and medical image data or subsets of it. In connection with FIGS. 1-3, the interaction of some example components of possible embodiments of this computer system have already been described schematically. At this point, the structure of some possible embodiments will be further discussed.

Some embodiments comprise at least one processor, at least one database and at least one memory. The memory stores, among other things, instructions that are executed by the at least one processor which provides the computer system with means for generating structured medical findings reports. This can be done, for example, in the form of the reporting environment 101 shown in FIG. 1. Furthermore, means are provided for displaying medical image data, which may be done, for example, in the form of the image display environment 103. Means are provided for recording a creation time, that is, a time attributable to the creation of a findings report entry in a structured medical findings report. Further, means are provided for determining medical image data or subsets of it displayed within a predetermined time period attributable to the recorded time of creation. Additionally, or alternatively, these means may also determine image identification data that may be associated with the displayed medical image data. Finally, means are provided for associating the previously determined image data/image identification data and the created findings report entry and means for automatically storing the associations is in a database. In some embodiments, the means described above for detecting a creation time, for determining image data and/or image identification data, for associating the image data/image identification data and the findings report entry with each other, and for automatically storing the associations in a database are partially or completely bundled in software or hardware, for example in the form of a hub 105 described above or as parts of the findings report reporting environment 101 and/or the image display environment 103.

The at least one processor is further adapted to implement associations of image data/image identification data and findings report entries as described in connection with the corresponding embodiments described above. Further, the at least one processor may optionally be configured to store the associations in one or more databases as described in connection with the corresponding embodiments already described.

Another aspect of the invention relates to a computer system for providing medical reference image data, and, optionally, further medical reference data, during the generation of a structured medical findings report on a patient including one or more medical findings report entries. In some embodiments, the computer system includes at least one processor, at least one database that was generated in one of the ways described earlier and at least one memory. The memory stores, among other things, instructions that are executed by the at least one processor. As a result, means for generating structured medical findings reports, for example in the form of the reporting environment 101, are provided by the computer system. This may be done in the image display environment 103 in some embodiments, and in one or more further image display environments in other embodiments. In addition, means are provided for providing an automatic or semi-automatic search for medical reference image data. In some embodiments, this search is provided directly when a findings report entry is created in a structured medical findings report. The search itself is performed in the at least one database. Additionally, in some embodiments, information obtainable from the structured medical findings report, in particular from a medical findings report entry that is related to findings in first medical image data of the patient is used for the search, e.g., as input parameters. The search identifies, based on the input parameters, subsets of the second medical image data to provide them as output medical image data. Finally, means for providing the output medical image data as reference image data are provided. In some embodiments, the system further comprises means for displaying medical image data in which the medical reference image data determined by the search are displayed.

Further embodiments of the methods and systems discussed include both executing the method for associating findings report entries of a structured medical findings report and medical image data, and executing the method for providing medical reference image data during the generation of a structured medical findings report. In the relevant methods and systems, the association of findings report entries and medical image data discussed above is performed during the generation of structured medical findings reports, and, if applicable, the findings report entries and medical image data are stored in one or more databases in one of the ways described above, and a search for medical reference data is provided simultaneously.

For example, the search may access the same database that may be populated during the generation of the present findings report as described. Thus, in some embodiments, the user may report on medical image data in a structured medical findings report using a reporting environment. While creating findings report entries the relevant medical image data is associated with the created finds entry in one of the above-described manners. In addition, the user may use a search function for inspecting relevant reference medical image data, and, maybe also further reference medical data as described in one of the above embodiments and examples. The search starts a query in a database that was created and/or filled as described in one of the above-described manners. Based on the guidance by the reference data, the user enters a new findings report entry which is automatically associated with the medical image data displayed in the image display environment while the new findings report entry was created. Eventually, the association is stored in the above database.

A further aspect of the invention relates to a computer program product comprising instructions for execution on at least one processor that, when executed, cause the processor to perform the steps of any one of the methods discussed above.

A further aspect of the invention relates to a computer-readable storage medium on which the above computer program product is stored.

A further aspect of the invention relates to a data carrier signal comprising the above computer program product.

1. A computer-implemented method i) for associating findings report entries of a structured medical findings report concerning medical image data and comprising at least one findings report entry, and one or more subsets of the medical image data, and ii) for generating a database comprising associations of findings report entries of structured medical reports and one or more subsets of medical image data, the method including steps comprising:
a. displaying subsets of the medical image data in an image display environment;
b. creating, on the user side, a medical findings report entry in the structured medical findings report and automatically recording a creation time attributable to the creation of the medical findings report entry;
c. automatically determining one or more subsets of the medical image data displayed within a predetermined time period attributable to the recorded creation time;
d. automatically associating the one or more subsets of the medical image data determined in step c), or image identification data enabling the determined one or more subsets of the medical image data to be located, and the created medical findings report entry;
e. automatically storing the association made in step d) in a database.

2. The method of claim 1, wherein the subsets of the medical image data are at least one of one or more slices of a set of tomographic image data, one or more regions of interest in one or more medical images or one or more parts of an electrogram.

3. The method of claim 1, wherein, in step c), additionally, setting parameters used for displaying the determined one or more subsets of the medical image data in the image display environment are automatically determined, and, in step d), additionally, the determined setting parameters are automatically associated with the created findings report entry.

4. The method of claim 1, wherein automatically storing the association in the database further includes storing at least one of the structured medical findings report, the findings report entry, findings report identification data enabling the findings report entry to be located, a findings identifier, and an ontology code identifying the findings; and wherein at least one of the determined one or more subsets of the medical image data associated with the findings report entry or image identification data enabling the determined one or more subsets of the medical image data to be located.

5. The method of claim 1, wherein automatically storing the association in the database further includes storing the setting parameters used for displaying the determined one or more subsets of the medical image data in the image display environment associated with the medical findings report entry.

6. A computer-implemented method for providing medical reference image data during the generation of a first structured medical findings report on a patient including one or more medical findings report entries, the computer-implemented method comprising:
a. providing a reporting environment for creating structured medical findings reports;
b. providing an automatic or semi-automatic search for reference medical image data during the creation of a medical findings report entry of the first structured medical findings report, wherein the medical findings report entry is related to findings in first medical image data of the patient and wherein the search uses as input parameters information retrievable from the medical findings report entry;
wherein the search is performed in a database generated using at least the steps of:
displaying subsets of second medical image data in an image display environment;
creating, on the user side, a medical findings report entry in a second structured medical findings report and automatically recording a creation time attributable to the creation of the medical findings report entry;
automatically determining one or more subsets of the second medical image data displayed within a predetermined time period attributable to the captured creation time;
automatically associating the one or more subsets of the second medical image data determined in step c) or image identification data enabling the determined one or more subsets of the second medical image data to be located, and the created medical findings report entry;
automatically storing the association made in step d) in a database; and,
based on the input parameters, identifies in the database subsets of the second medical image data to provide them as output medical image data; and
c. providing the output medical image data as reference image data.

7. (canceled)

8. The method of claim 6, wherein the provided automatic or semi-automatic search additionally searches for reference medical findings reports associated with the reference medical image data.

9. The method of claim 6, additionally comprising the steps of:
i. displaying subsets of the medical image data in an image display environment;
ii. creating, on the user side, a medical findings report entry in the structured medical findings report and automatically recording a creation time attributable to the creation of the medical findings report entry;
iii. automatically determining one or more subsets of the medical image data displayed within a predetermined time period attributable to the recorded creation time;
iv. automatically associating the one or more subsets of the medical image data determined in step iii), or image identification data enabling the determined one or more subsets of the medical image data to be located, and the created medical findings report entry;
v. automatically storing the association made in step iv) in a database.

10. A computer system for associating medical findings report entries of a structured medical findings report concerning medical image data and comprising at least one medical findings report entry, and one or more subsets of the medical image data, and for generating a database comprising associations of findings report entries of structured medical reports and one or more subsets of medical image data, the computer system comprising:
at least one processor;
at least one database; and
at least one memory having stored thereon instructions which, when executed on the at least one processor, cause the computer system to provide:
means for creating structured medical findings reports;
means for displaying medical image data;
means for recording a creation time attributable to the creation of a medical findings report entry in the structured medical findings report;
means for determining one or more subsets of the medical image data displayed within a predetermined time period attributable to the recorded creation time; and
means for associating the determined one or more subsets of the medical image data or image identification data enabling the determined subsets of the medical image data to be located, and the created medical findings report entry means for automatically storing the associations in a database.

11. A computer system for providing medical reference image data during the generation of a first structured medical findings report on a patient including one or more medical findings report entries, comprising:
at least one processor;
at least one database generated using at least the following steps:
a) displaying subsets of the medical image data in an image display environment;
b) creating, on the user side, a medical findings report entry in a second structured medical findings report and automatically recording a creation time attributable to the creation of the medical findings report entry;
c) automatically determining one or more subsets of the second medical image data displayed within a predetermined time period attributable to the captured creation time;
d) automatically associating the one or more subsets of the second medical image data determined in step c) or image identification data enabling the determined one or more subsets of the second medical image data to be located, and the created medical findings report entry;
e) automatically storing the association made in step d) in a database; and
at least one memory having stored thereon instructions which, when executed on the at least one processor, cause the computer system to provide:
means for creating structured medical findings reports;
means for providing an automatic or semi-automatic search for medical reference image data when creating a medical findings report entry of the first structured medical findings report, wherein the medical findings report entry is related to findings in first medical image data of the patient, wherein the search uses as input parameters information retrievable from the medical findings report entry is performed in the at least one database and, based on the input parameters, identifies in the database subsets of the second medical image data to provide them as output medical image data; and
means for providing the output medical image data as reference image data.

12. The computer system of claim 11, wherein the subsets of the medical image data are at least one of one or more slices of a set of tomographic image data, one or more regions of interest in one or more medical images, or one or more parts of an electrogram.

13-15. (canceled)

16. The computer system of claim 11, wherein the computer system is further caused to automatically determine and set parameters used for displaying the determined one or more subsets of the medical image data in the image display environment, and automatically associate determined setting parameters with the medical findings report entry.

17. The computer system of claim 11, wherein automatically storing the association in the database further includes storing at least one of the structured medical findings report, the findings report entry, findings report identification data enabling the findings report entry to be located, a findings identifier, and an ontology code identifying the findings, and wherein at least one of the determined one or more subsets of the medical image data associated with the findings report entry or image identification data enables the determined one or more subsets of the medical image data to be located.

18. The computer system of claim 11, wherein automatically storing the association in the database further includes storing the setting parameters used for displaying the determined one or more subsets of the medical image data in the image display environment associated with the medical findings report entry.

19. A non-transitory, computer-readable storage medium having instructions stored thereon that, when executed by a computer processor, cause the computer processor to carry out a process for providing medical reference image data during the generation of a first structured medical findings report on a patient including one or more medical findings report entries, the process comprising:
generating a database using at least the following steps:
a) displaying subsets of the medical image data in an image display environment;
b) creating, on the user side, a medical findings report entry in a second structured medical findings report and automatically recording a creation time attributable to the creation of the medical findings report entry;
c) automatically determining one or more subsets of the second medical image data displayed within a predetermined time period attributable to the captured creation time;
d) automatically associating the one or more subsets of the second medical image data determined in step c) or image identification data enabling the determined one or more subsets of the second medical image data to be located, and the created medical findings report entry;
e) automatically storing the association made in step d) in a database; and
creating structured medical findings reports;
providing an automatic or semi-automatic search for medical reference image data when creating a medical findings report entry of the first structured medical findings report, wherein the medical findings report entry is related to findings in first medical image data of the patient. wherein the search uses as input parameters information retrievable from the medical findings report entry is performed in the at least one database; and, based on the input parameters, identifies in the database subsets of the second medical image data to provide them as output medical image data; and
providing the output medical image data as reference image data.
